(19) United States
(12) Patent Application Publication
(10) Pub. No.: US 2008/0051746 A1
(43) Pub. Date: Feb. 28, 2008

(54) SURGICAL SPONGE INCORPORATING RFID TECHNOLOGY AND METHOD OF USE

(76) Inventor: Jane Shen-Gunther, Dupont, WA (US)

Correspondence Address: GREENBERG TRAURIG, 3773 HOWARD HUGHES PARKWAY, SUITE 500 NORTH, LAS VEGAS, NV, 89169, US

(21) Appl. No.: 11/466,746

(22) Filed: Aug. 23, 2006

(51) Int. Cl. A61F 13/15

(52) U.S. Cl. 604/362

(57) ABSTRACT

A surgical sponge with an integrated RFID transponder is disclosed. A laundry RFID transponder integrated between layers of the sponge provides means for surgical personnel to verify with a RFID reader that all such sponges have been removed from a patient's body prior to an incision being closed or from a body cavity without an incision. The RFID reader may also be used post closure to re-verify that no such sponges have been inadvertently closed within the patient. A system may include a storage means and associated display device to allow for easy and convenient verification that all sponges made available for a particular surgery have all been accounted for prior to the surgical incision being closed.

### FIELD OF THE INVENTION

The embodiments of the present invention relate to the use of RFID technology for tracking the location of surgical sponges.

### BACKGROUND

Incidents of surgical instruments and related items being left in patients are well documented. Such incidents cause health complications for the subject patients and create liability issues for the medical staff. One particularly problematic item is the surgical sponge. Surgical sponges, for soaking blood and other bodily fluids, are ubiquitously used during surgical procedures. With so many sponges being used, they are difficult to track during surgical procedures. Therefore, sponges are all too often the item left in a patient after a wound has been closed.

The use of Radio Frequency Identification (RFID) technology is being used to track all types of products from manufacture to wholesaler to distributor to retailer to consumer. RFID technology has improved over time such that it can now be used to track other items, like surgical sponges, used in more harsh conditions than conventional consumer products.

Thus, there exists a need for a RFID-based system for tracking surgical sponges and preventing them from being left in patients after the closure of wounds.

### SUMMARY

Accordingly, one embodiment of the present invention is a surgical item comprising: a surgical sponge for use during surgical procedures; a RFID transponder integrated within said sponge, said RFID transponder capable of functioning after enduring fluids, including blood and other bodily fluids.

A method of tracking surgical sponges according to one embodiment of the present invention comprises: integrating a RFID transponder into a plurality of surgical sponges; recording use of the sponges by reading a transponder signal and transmitting the use of the sponges to a storage means and associated display device; after need for the sponges has ceased, removing the sponges from a patient and recording non-use of the sponges by reading the transponder signal and transmitting its non-use of the sponges to the storage means and associated display device; and comparing the use data of the sponges with the non-use data of the sponges to confirm that all used sponges have been identified and removed from the patient.

A system for tracking surgical sponges according to one embodiment of the present invention comprises: one or more surgical sponges, each of the one or more sponges including a RFID transponder: a RFID scanner or reader operable to identify each sponge by reading a RFID transponder associated with the sponge; and a storage device for recording the identification acquired by the RFID scanner or reader.

Other variations, embodiments and features of the present invention will become evident from the following detailed description, drawings and claims.

### BRIEF DESCRIPTION OF THE DRAWINGS

FIG. 1 illustrates a surgical sponge;

FIG. 2 illustrates a RFID transponder of the type that may facilitate the embodiments of the present invention;

FIG. 3 illustrates a surgical sponge with a RFID transponder integrated therein;

FIG. 4 illustrates a hand-held RFID scanner/reader; and

FIGS. 5a-c illustrate various exemplary screen shots showing a tracking procedure for surgical sponges.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

The embodiments of the present invention utilize RFID technology. RFID technology is not new such that the minute details of how RFID technology works are not disclosed herein as those skilled in the art will recognize the technology. Notwithstanding the foregoing, this disclosure provides the requisite information to allow one skilled in the art to practice the invention.

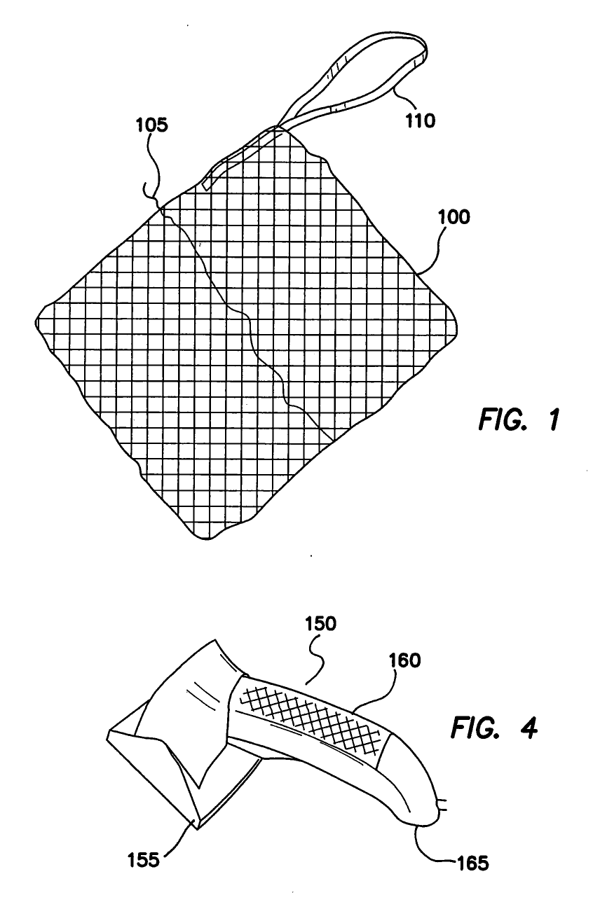

Initial reference is made to FIG. 1 illustrating a perspective view of a surgical sponge generally identified by reference numeral 100. More particularly, FIG. 1 shows a laparotomy sponge that in practice is used for staunching bleeding and for wound treatment, and for gripping and retaining organs and tissue during surgery. The sponge 100 includes a sewn in loop 110 for handling the sponge 100 and a radio-opaque member 105 to show up on X-ray. Laparotomy sponges comprise many layers of gauze-like material. While a laparotomy sponge is shown, any type of surgical sponge may be utilized with the embodiments of the present invention. The rate at which surgical sponges are retained in a patient is not directly quantifiable, but suffice it to say the rate is much higher than preferred.

Studies suggest that the retention of sponges is not a random event but rather is more likely to occur 1) during emergency surgery; 2) when there is an unexpected change in surgical procedure; and 3) in obese patients. A retained sponge creates numerous patient complications ranging from subsequent surgery, infections and death and further creates legal liability for the hospital, emergency center and/or surgeons where the sponge is left in the patient. Traditionally, procedures have been enlisted to prevent surgical instruments and sponges from being retained in patients. For example, sponges should be counted on all procedures in which the possibility exists that a sponge could be retained. Sponge counts should be taken: 1) before the procedure to establish a baseline. 2) before closure of a cavity: 3) before wound closure begins; 4) at skin closure or end of procedure; and 5) at the time of permanent relief of either the scrub person or a nurse. The disclosed procedure and others obviously are not fail-proof and require a dedicated staff.

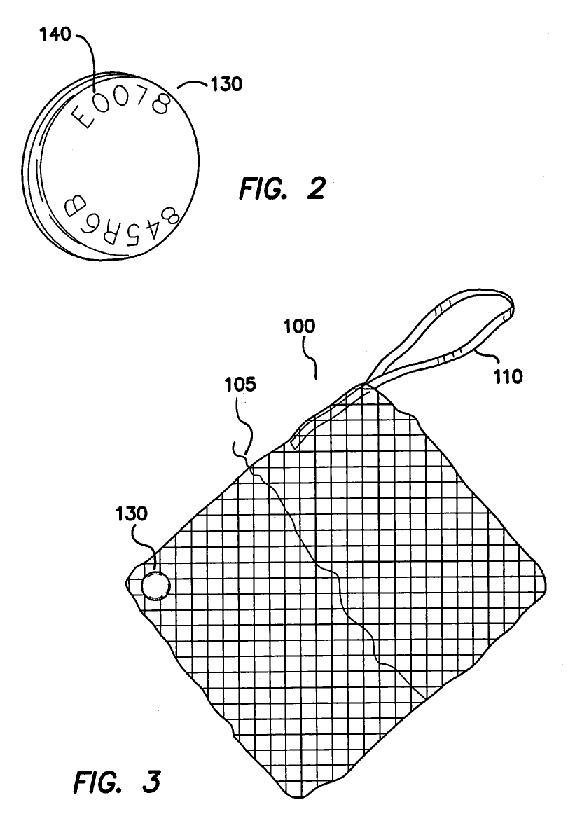

FIG. 2 shows a RFID tag or transponder 130 that may be used to facilitate a first embodiment of the present invention. The RFID transponder 130 comprises a microchip and antenna. Importantly, the RFID tag 130 used to facilitate the embodiments of the present invention must be able to endure blood and other bodily fluids while being anti-collision, sterilization resistant against gamma radiation or ethylene oxide (ETO) which are used to sterilize sponges, read and write capable and inert in the human body. The anti-collision criteria allows multiple sponges to be identified simultaneously. As shown in FIG. 2, one suitable RFID transponder is a laundry RFID transponder 130 manufactured by Texas Instruments. In one embodiment, the transponder 130 is a 13.56 MHz passive laundry transponder designed for harsh environments. The transponder 130 includes a laser etched unique identification number 140 on the transponder housing. Other RFID transponders are fabricated with glass housings and may be suitable for the embodiments of the present invention. As shown in FIG. 3, ideally the RFID transponder 130 is sewn or thermally attached to a subject sponge as represented by the dotted circle 140. Any suitable, secure means of attachment may be used.

In a RFID system of the present invention, individual sponges, like sponge 100, are equipped with a RFID transponder 130. The RFID transponder 130 includes a digital memory chip that is given a unique electronic product code. An interrogator or scanner/reader 150 (shown in FIG. 4), comprising an antenna packaged with a transceiver and decoder, emits a signal activating the RFID transponder 130 so it can read and write data to it. The reader 150 is able to decode the data encoded in an integrated circuit (silicon chip) of the RFID transponder 130 and the data is passed to a host system, such as a computer. Application software running on the host system processes the data. In one embodiment, the application software utilizes Physical Markup Language (PML). However, in other embodiments, other languages may be used. One suitable scanner is the OBID iScan manufactured by FEIG Electronic GmbH.

Now referring to FIG. 4, the scanner/reader 150 comprises a base member 155, elongated member 160 and a reader end 165. The reader 150 may transmit the data to the host system via a wired or wireless connection. Suitable readers may take on any shape and configuration, including hand-held variations, such as PDAs, to facilitate the embodiments of the present invention.

In one practical system example, the subject surgical sponges 100 are fitted with RFID transponders 130. Transponder data corresponding to the identity and location of each RFID transponder 130 are maintained in a stored database controlled by the host system. Users are able to view the database information on a system screen like a computer monitor or the like. Thus, each sponge that is intended for use in a surgery is entered into the database as such. The reader 150 is utilized to enter each sponge as active into the database. Then, after the surgery is complete and, ideally, before the surgical incision is closed, each sponge is passed by and deactivated by means of the reader 150. The host system then determines whether all activated sponges are now deactivated thereby confirming that all sponges have been verified outside of the patient such that the incision may be closed safely.

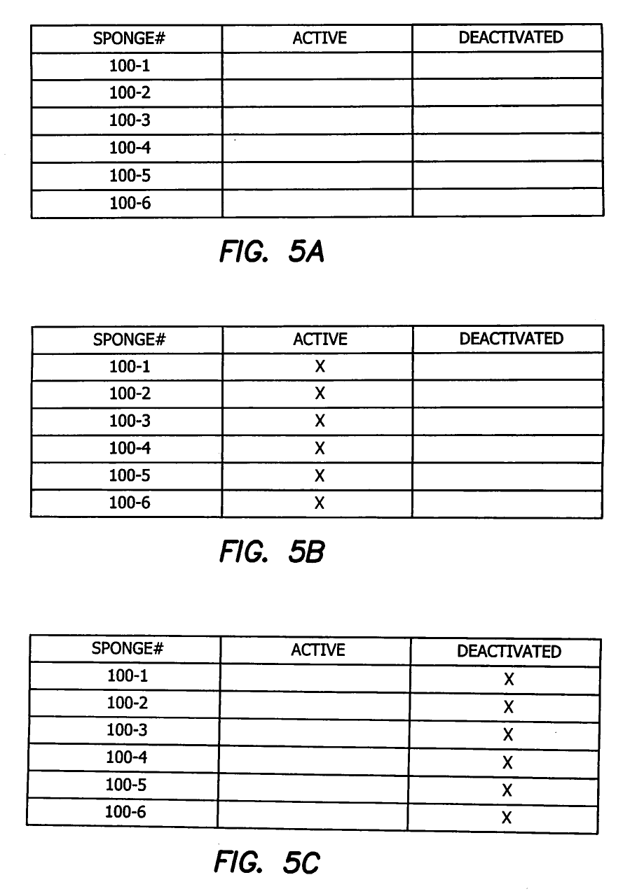

FIGS. 5a-c show a host system display 200 at various times during the surgery procedure. Initially, as shown in FIG. 5a, the display 200 depicts a table 205 having columns listing sponge identification codes 201, and sponge status as active (i.e., in use) 202 or deactivated (i.e., not in use) 203. Then, once the subject sponges have been collected for a particular surgical procedure, the sponges are entered into the host system as active. Accordingly, in FIG. 5b, the display 200 shows six sponges 100-1 through 100-6 as active. Then, as shown in FIG. 5c, after each sponge has been used or the surgical procedure ends, the sponges 100-1 through 100-6 are entered as deactivated. The host system then verifies, and the user can visualize, that all sponges 100-1 through 100-6 that were originally activated are now deactivated indicating that all sponges have been removed from the patient or were not used.

Besides using sponges within incisions, they may also used in vaginal delivery, ear-nose-throat procedures or anorectal surgeries which are technically entries into a body cavity and do not always require an incision. The embodiments of the present invention are just as suitable in these body cavity procedures.

The use of the laundry RFID transponder 130 permits the system to function properly as the laundry RFID transponder 130 is able to endure and continue to function after blood and other bodily fluids are encountered during use.

Although the invention has been described in detail with reference to several embodiments, additional variations and modifications exist within the scope and spirit of the invention as described and defined in the following claims.

1. A surgical item comprising:
a surgical sponge for use during surgical procedures; and
a RFID transponder integrated within said sponge, said RFID transponder capable of functioning after enduring fluids, including blood and other bodily fluids.

2. The surgical item of claim 1 wherein the sponge is a laparotomy sponge.

3. The surgical item of claim 1 wherein the sponge includes a radio-opaque member.

4. The surgical item of claim 1 wherein the RFID transponder is thermally attached to, or sewn into, the sponge.

5. A surgical system comprising:
a surgical sponge;
a laundry RFID transponder integrated between two layers of the sponge; and
a RFID reader operable to identify the RFID transponder.

6. The system of claim 5 wherein the RFID reader is a hand held device.

7. The system of claim 5 wherein the surgical sponge is a laparotomy sponge.

8. A surgical method comprising:
performing a surgical procedure requiring an incision, closing of the incision and the use of one or more surgical sponges;
completing the surgical procedure except for closing the incision;
using a RFID reader to confirm that each of the one or more sponges is outside of a patient's body; and
closing the incision.

9. The method of claim 8 wherein the RFID reader is a hand held device.

10. The method of claim 8 further comprising using the RFID reader after closing the incision to re-confirm that each of the one or more sponges is outside of a patient's body.

11-12. (canceled)

13. A method of tracking surgical sponges, comprising:
integrating a RFID transponder into a plurality of surgical sponges;
recording use of the sponges by reading a transponder signal and transmitting the use of the sponges to a storage means and associated display device;
after need for the sponges has ceased, removing the sponges and recording non-use of the sponges by reading the transponder signal and transmitting its non-use of the sponges to the storage means and associated display device; and
comparing the use data of the sponges with the non-use data of the sponges to confirm that all used sponges have been identified and removed from the patient.

14. The method of claim 13 wherein the storage means comprises a database maintained on a computer system.

15. A system for tracking surgical sponges comprising:
one or more surgical sponges, each one or more sponge including a RFID transponder;
a RFID scanner or reader operable to identify each sponge by reading a RFID transponder associated with the sponge; and
a storage device for recording the identification acquired by the RFID scanner or reader.

16. The system of claim 15 wherein the RFID scanner or reader is a hand-held device.

17. The system of claim 15 further comprising a display device in communication with the storage device.
